(19) United States
(12) Patent Application Publication
(10) Pub. No.: US 2024/0374769 A1
(43) Pub. Date: Nov. 14, 2024

(54) LIGHT ACTIVATED CLEANING COMPOSITION

(71) Applicant: The Procter & Gamble Company, Cincinnati, OH (US)

(72) Inventors: Yonas GIZAW, West Chester, OH (US); Roy Jerome HARRINGTON, Liberty Township, OH (US); Nathaniel OKPOSIO, Fairfield, OH (US); Stephan Gary BUSH, Liberty Township, OH (US); Faiz Feisal SHERMAN, Mason, OH (US); Francisco RODRIGUEZ ROPERO, South Lebanon, OH (US)

(21) Appl. No.: 18/656,643

(22) Filed: May 7, 2024

Related U.S. Application Data
(60) Provisional application No. 63/501,045, filed on May 9, 2023.

(51) Int. Cl. A61L 2/08; A61L 2/18; A61L 101/44

(52) U.S. Cl. CPC A61L 2/088; A61L 2/18; A61L 2101/44; A61L 2202/26

(57) ABSTRACT

A light activated cleaning composition comprising a photosensitizer and a singlet oxygen quenching molecule.

### FIELD OF THE INVENTION

The present invention relates to the cleaning of fabrics without the need for a conventional dry cleaning or aqueous wash process, using light activated cleaning compositions.

### BACKGROUND OF THE INVENTION

The regular washing of fabrics, such as clothes, bed sheets, and other household items, via traditional laundry processes is very effective; however, conventional immersive cleaning and tumble-drying processes utilize extensive resources (water, energy), time and may result in mechanical damage to fabrics via the agitation/tumbling processes used in laundry and drying machines. Further, the chemistries used in cleaning such as bleach, surfactants can degrade fabrics, thus shortening the life-span of an article. Further not all worn fabrics need a full wash cycle to be usable after wear, for instance they may have some moderate soilage which could be remedied using cleaning and refreshing actions other than a full wash cycle.

During regular use it is common for fabrics to come into contact with various soils which can cause staining and malodor. The soils can make the fabric unusable, leaving an individual with the choice of either not using the article or submitting it to a cleaning process. In addition, most cleaning processes take an extended period of time requiring an aqueous wash cycle followed by a drying process or in the case of non-aqueous processes (“dry-cleaning”) an even longer time until the article is once again ready for use, as a professional cleaner is often needed.

There is a need for a fabric cleaning process that has a reduced clean time compared to current washing systems, and that reduces negative effects caused by washing, such as bleaching and fraying.

### SUMMARY OF THE INVENTION

A light activated cleaning composition is provided that comprises a photosensitizer; and a singlet oxygen quenching molecule.

A method of cleaning a fabric is provided that comprises applying a light activated cleaning composition comprising Riboflavin 5-phosphate sodium salt and imidazole to a fabric; exposing the light activated cleaning composition to light; and generating at least one of endoperoxide or exoperoxide or hydrogen peroxide.

A light activated cleaning composition is provided that comprises a photosensitizer; and sodium Ascorbate.

### BRIEF DESCRIPTION OF THE DRAWINGS

While the specification concludes with claims particularly pointing out and distinctly claiming the subject matter that is regarded as the present disclosure, it is believed that the disclosure will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of elements in any of the exemplary embodiments, except as may be explicitly delineated in the corresponding written description. None of the drawings are necessarily to scale.

FIG. 1 is an illustration of a photosensitizer mode of action.

FIG. 2 is an illustration showing determination of photobleaching efficiency.

FIG. 3 is a graph showing depletion of thioxanthone 23 μM in ethanol with 365 nm irradiation

FIG. 4 is a graph showing a rate of decay of N,N-dimethyl-4-nitrosoaniline visible marker (NA) as detected by following it's absorbance at 424 nm.

FIG. 5 is a graph showing the impact of sodium ascorbate (SA) on Riboflavin 5-phosphate sodium salt (RF) degradation.

### DETAILED DESCRIPTION OF THE INVENTION

The light activated cleaning compositions (LACC) of the present invention comprise a photosensitizer capable of producing reactive oxygen species (ROS) and a compound that reacts with singlet oxygen to produce at least one of endoperoxide(s), exoperoxide(s), or hydrogen peroxide. Two examples of LACCs comprise (1) Riboflavin5-phosphate and inidazole and (2) thioxanthone and sodium ascorbate.

As used herein, the term “biodegradable components” refers to components that have the capacity to be biologically degraded by living organisms down to the base substances such as water, carbon dioxide, methane, basic elements and biomass. “Biodegradability” can be assessed according to OECD readily biodegradable 301 test protocols, preferably the OECD 301B test protocol (OECD (1992), Test No. 301: Ready Biodegradability, OECD Guidelines for the Testing of Chemicals, Section 3, OECD Publishing, Paris, https://doi.org/10.1787/9789264070349-en.).

As used herein, the term “renewable component” refers to a component that is derived from renewable feedstock and contains renewable carbon. A renewable feedstock is a feedstock that is derived from a renewable resource rather than being geologically derived. A material may be partially renewable (less than 100 percent renewable carbon content, from about 1 percent to about 90 percent renewable carbon content, or from about 1 percent to about 80 percent renewable carbon content, or from about 1 percent to about 60 percent renewable carbon content, or from about 1 percent to about 50 percent renewable carbon content) or can be 100 percent renewable (100 percent renewable carbon content). A renewable feedstock may be blended or chemically reacted with a geologically derived feedstock, resulting in a material with a renewable component and a geologically derived component. The renewable carbon content can comprise one or more of the following or mixtures thereof:
(a) Ingredients fully or partly derived from biomass, include food crops, non-food crops, side streams, by-products and biogenic waste;
(b) Ingredients that are fully or partly bio-based;
(c) Ingredients that are fully or partly made by carbon capture and usage (CCU) wherein said captured carbon can be carbon dioxide or methane;
(d) Ingredients that are fully or partly mechanically or chemically recycled (e.g., plastic, tires);
(e) Ingredients that are fully or partly made by conversion of waste chemicals such as plastic or rubber pyrolysis.

As used herein, the term “biomass derived” refers to materials derived from a first, second or third generation biomass. First generation biomass is derived from vegetable oil, starch or sugar coming from an existing row crop. Second generation biomass is derived from cellulosic biomass sources including crop residues, rotational crops, perennial grasses, forestry residues, waste oils and fats such as used cooking oils (UCO), animal fats, crude tall oil (CTO), or Distillers Corn Oil (DCO) and trees. Second generation biomass sources may be grown on marginal cropland where row crop production is not profitable. By focusing on areas that are highly erodible or have marginal soil quality, this avoids competition with fertile ground that may be best used to grow food crops. Third generation biomass or oil includes oils harvested from algae and solid waste biomass.

As used herein, the term “bio-based” refers to the feedstock that a material is made from. Biobased materials are made from renewable (non-petroleum) based sources (plants and animals and/or micro-organisms). Some plants that are used to make bio-based materials include sugarcane, cassava, soy-beans, sugar beets, palm, coconut, rapeseed, canola, algae, switchgrass, camelina, macauba, carinata and corn. Some materials are partially biobased by combining fossil-based and biobased components into one material. Bio-naphtha and advanced fuels such as bio-diesel and sustainable aviation fuel represent bio-based feedstocks that can be utilized and converted into useful materials including surfactants, solvents and polymers. Bio-based content can be measured using the “Assessment of the Biobased Content of Materials” ASTM D6866-16 test method.

The term “fabrics” and “fabric” used herein is intended to mean any article that is customarily cleaned in a conventional laundry process or in a dry-cleaning process. As such the term encompasses articles of clothing, linen, drapery, and clothing accessories. The term also encompasses other items made in whole or in part of fabric, such as tote bags, furniture covers, tarpaulins and the like.

The term “soil” means any undesirable substance on a fabric. By the terms “water-based” or “hydrophilic” soils, it is meant that the soil comprised water at the time it first came in contact with the fabric article, or the soil retains a significant portion of water on the fabric article. Examples of water-based soils include, but are not limited to beverages, many food soils, water soluble dyes, bodily fluids such as sweat, urine or blood, outdoor soils such as grass stains and mud.

The term “malodor” as used herein is most commonly caused by environmental odors such as tobacco odor, cooking and/or food odors, or body odor. The unpleasant odors are mainly organic molecules which have different structures and functional groups. One type of malodor that is very noticeable and is commonly found on worn fabrics is low molecular weight, straight-chain, branched, and unsaturated C₆-C₁₁ fatty acids that cause axillary odor. See “Analysis of Characteristic Odor from Human Male Axillae”. X. Zeng, et al., J. Chem. Ecol., pp. 1469-1492, 1991, incorporated herein by reference. See also. U.S. Pat. No. 4,664,909, Marschner et al., issued May 12, 1987, BE 830, 098, published Oct. 1, 1975, and CA 1, 088,428, published Oct. 28, 1980, DE 2,503,176, published Aug. 3, 1978.

All temperatures herein are in degrees Celsius (° C.) unless otherwise indicated. Unless otherwise specified, all measurements herein are conducted at 20° C. and under the atmospheric pressure.

As used herein, the articles “a” and “an” when used in a claim, are understood to mean one or more of what is claimed or described.

As used herein, the terms “include,” “includes,” and “including” are meant to be non-limiting.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

In some embodiments, suitable photosensitizer molecules are at least one of substituted or unsubstituted flavins, Acetonaphthones, Acridines, Anthracenes, Anthraquinones, Anthrones, Azulenes, Benzophenones, Benzoquinones, Flavones, Camphoroquinone, Chrysenes, substituted 7-Dehydrocholesterols, Ergosterols, Fluorenes, s Fluorenones, Eosins, Fluoresceins, Phloxines, Rose Bengals, Erythrosins, Indoles, Naphthalenes, Phenanthrenes, Phenazines, Thionines, Azures, Toluidine Blue, Methylene Blues, Pyrenes, Quinoxalines, Retinols, Riboflavins, Rubrenes, Bacteriochlorophylls, Chlorophylls, Pheophytins, Pheophorbides, Protochlorophylls, protoporphyrins, Fullerenes, Porphyrins, Metallo Porphyrins, Porphines, Rubrenes, and Phthalocyanines, phenosafranine, Thiamine hydrochloride, 2-((6,11-Dihydro-5H-dibenzo[b,e]azepin-6-yl)methyl)isoindoline-1,3-dione, Rivaroxaban Impurity 9, Warfarin sodium, Indomethacin, Dimethylaminoethyl acrylate benzyl chloride, Riboflavin sodium phosphate, (3-Acrylamidopropyl)trimethylammonium Chloride, 2,2′-Bis(2-chlorophenyl)-4,4′,5,5′-tetraphenyl-1,2′-biimidazole, 7-(4-(Diethylamino)-2-ethoxyphenyl)-7-(1-ethyl-2-methyl-1H-indol-3-yl)furo[3,4-b]pyridin-5(7H)-one, Victoria Blue R, Ethalfluralin, N-(6-chloro-4-methylbenzo[d]thiazol-2-yl)-4-cyano-N-(2-morpholinoethyl)benzamide hydrochloride, Curcumin, Zincon, 2-Mercaptonicotinic acid, N-(cyclohexylcarbamothioyl)benzamide, Ninhydrin, Tolperisone, 3H-Indolium, 1,3,3-trimethyl-2-[2-(1-methyl-2-phenyl-1H-indol-3-yl)ethenyl]-, chloride (1:1), Chloropheniramine maleate, 3,3′-Carbonylbis(7-diethylaminocoumarin), Methyl (triphenylphosphoranylidene)acetate, 1 h-Pyrrole-2,5-dione, 1,1′-[1,2-ethanediylbis(thio-2,1-phenylene)]bis-1-Propanone, 1-(1,1′-biphenyl)-4-yl-2-methyl-2-(4-morpholinyl)-N-(2-chlorophenyl)-N′-{[2-(3-isopropoxyphenyl)-4-quinolinyl]carbonyl}thiourea, 2(1H)-Quinazolinethione, 4-(2-propen-1-ylamino)-, Benidipine Ethyl 2-amino-4-methyl-5-(4-nitrophenyl)thiophene-3-carboxylate, Irgacure 369 Monophosphothiamine, N-Benzyl Salbutamon Hydrochloride, Girard P reagent, Dimethyl(2-((2-methyl-1-oxoallyl)oxy)ethyl)(3-sulphopropyl)ammonium hydroxide, N-(2-Methyl-5-nitrophenyl)-4-(pyridin-3-YL)-1,3-thiazol-2-amine, or their derivatives.

Riboflavin includes any flavin compound or analog thereof comprising a flavin moiety capable of generating singlet oxygen when activated by a light source of an appropriate wavelength. The term “riboflavin” includes the natural chemical riboflavin, also known as vitamin B2, as well as chemical analogs of riboflavin which are also capable of producing one or more oxygen free radicals and singlet oxygen upon application of a light source of appropriated wavelength. Also encompassed are conjugated forms comprising a flavin moiety, for example flavin adenine dinucleotide and flavin mononucleotide. Also included are solubilized versions of such compounds, for example derivatives including polar, charged, or other groups which improve solubility in predominantly aqueous media or compositions comprising a predominantly aqueous phase. An example of a solubilized form of riboflavin is riboflavin-5′-phosphate. In addition to flavin and derivative molecules that generate singlet oxygen upon light irradiation, the present invention includes Thiamine hydrochloride, 2-((6,11-Dihydro-5H-dibenzo[b,e]azepin-6-yl)methyl)isoindoline-1,3-dione, Rivaroxaban Impurity 9, Warfarin sodium, Indomethacin, Dimethylaminoethyl acrylate benzyl chloride, Riboflavin sodium phosphate, (3-Acrylamidopropyl)trimethylammonium Chloride, 2,2′-Bis(2-chlorophenyl)-4,4′,5,5′-tetraphenyl-1,2′-biimidazole, 7-(4-(Diethylamino)-2-ethoxyphenyl)-7-(1-ethyl-2-methyl-1H-indol-3-yl)furo[3,4-b]pyridin-5(7H)-one, Victoria Blue R, Ethalfluralin, N-(6-chloro-4-methylbenzo[d]thiazol-2-yl)-4-cyano-N-(2-morpholinoethyl)benzamide hydrochloride, Curcumin, Zincon, 2-Mercaptonicotinic acid, N-(cyclohexylcarbamothioyl)benzamide, Ninhydrin, Tolperisone, 3H-Indolium, 1,3,3-trimethyl-2-[2-(1-methyl-2-phenyl-1H-indol-3-yl)ethenyl]-, chloride (1:1), Chloropheniramine maleate, 3,3′-Carbonylbis(7-diethylaminocoumarin), Methyl (triphenylphosphoranylidene)acetate, 1 h-Pyrrole-2,5-dione, 1,1′-[1,2-ethanediylbis(thio-2,1-phenylene)]bis-1-Propanone, 1-(1,1′-biphenyl)-4-yl-2-methyl-2-(4-morpholinyl)-N-(2-chlorophenyl)-N′-{[2-(3-isopropoxyphenyl)-4-quinolinyl]carbonyl}thiourea, 2(1H)-Quinazolinethione, 4-(2-propen-1-ylamino)-, Benidipine Ethyl 2-amino-4-methyl-5-(4-nitrophenyl)thiophene-3-carboxylate, Irgacure 369 Monophosphothiamine, N-Benzyl Salbutamon Hydrochloride, Girard P reagent, Dimethyl(2-((2-methyl-1-oxoallyl)oxy)ethyl)(3-sulphopropyl)ammonium hydroxide, N-(2-Methyl-5-nitrophenyl)-4-(pyridin-3-YL)-1,3-thiazol-2-amine and their derivatives.

In some embodiments, compounds that produce endoperoxide(s) and/or exoperoxide(s) by reacting with singlet oxygen include but are not limited to conjugated olefins and olefins that contain at least one allylic hydrogen. Such molecules are referred to as “singlet oxygen quenching molecules.” Addition of singlet oxygen to diene systems can produce endoperoxides, either dioxetanes via 1,2-cycloaddition of singlet oxygen to an olefin or an endoperoxide via 1,4-cycloaddition. 1,3-addition of singlet oxygen to an allylic hydrogen yields an exoperoxide (i.e. an allylic hydroperoxide). Suitable compounds include substituted and unsubstituted imidazole, Benzimidazole, Indoles, pyrrole, furans, guanine, sodium ascorbate, histidine, nitrogen and oxygen-containing heterocycles and their derivatives.

Examples of endoperoxide and exoperoxide formation are shown below:

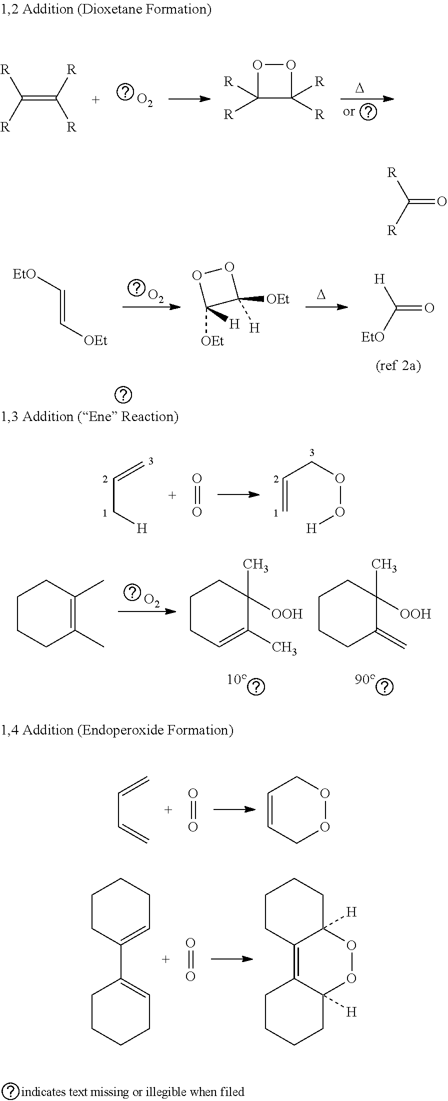

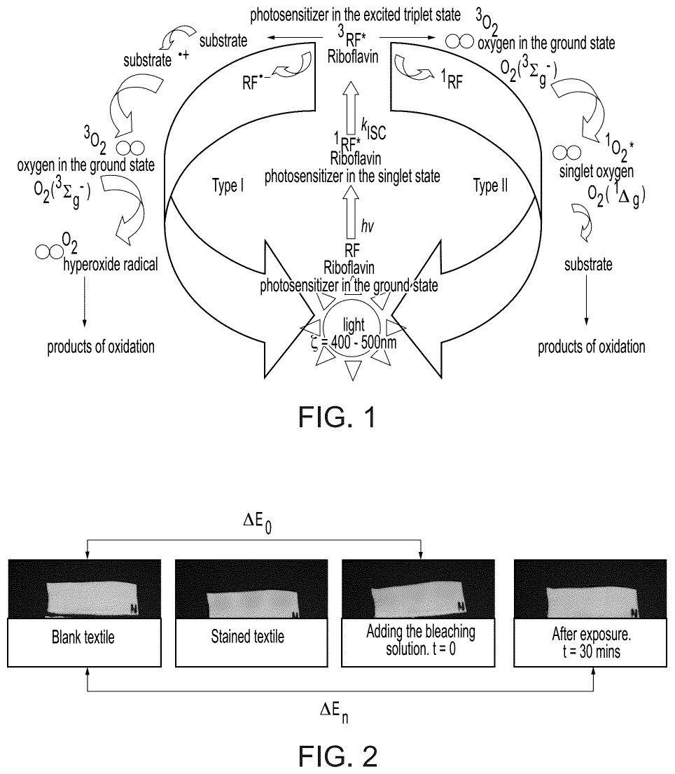

When applied to fabrics and exposed to light, the LACC provide stain and malodor reduction without the need for a detergent in either an aqueous or non-aqueous washing procedure. While not being bound by theory and as depicted in FIG. 1, when a photosensitizer molecule is exposed to light, the molecule absorbs energy and is excited to higher electronic energy states. Intersystem crossing can then populate other excited states, which enhances the possibility of the molecule of undergoing further reactions. In the presence of oxygen, energy transfer can produce either excited state triplet or singlet oxygen, both of which may either react with odor molecules to reduce malodors and/or bleach visible stains.

Free radicals can be formed in these reactions with molecular oxygen and electron transfers can then give other active forms of oxygen, like superoxide radical anions. These radical ions can also react with malodor molecules and provide stain and malodor reduction benefits.

The formation of singlet oxygen provides a high level of bleaching and odor reduction by reacting with dienes of the present invention. Singlet oxygen reaction with dienes, and heterocycles such as imidazole, furans, and their derivatives can produce both endo- and/or exo-peroxides. The combination of a photosensitizer with a singlet oxygen reacting molecule, such as an imidazole or furan derivative provides significant malodor reduction and visual stain removal advantages compared to using individual photosensitizer molecules alone.

An embodiment of the present invention includes the photosensitized generation of singlet oxygen in the presence of molecules such as sodium ascorbate to generate hydrogen peroxide to further deodorize and bleach stains on fabrics.

Both sunlight and artificial lights (i.e., both incandescent and/or fluorescent, LED) effectively cause a LACC to generate reactive oxygen and/or reactive addition product species.

Light sources useful for activating the photosensitizing molecules of the present invention include those capable of outputting visual and/or ultraviolet light of a wavelength and in an amount to create sufficient reactive oxygen species for the desired application. The light source can be a lamp, LED, laser, or a combination of sources. The light source may be filtered so as to produce a restricted wavelength or set of wavelengths of interest. In some cases, sunlight may be used as the light source. The light source may be focused, concentrated, or amplified.

Endo/exo peroxides and/or hydrogen peroxide may be generated by exposing the light activated cleaning compositions (LACC) described herein to light having a wavelength that can be absorbed by a photosensitizer in presence of dienes conjugated with heteroatoms, such as imidazole, furan, and their derivatives and/or hydrogen peroxide generators such as sodium ascorbate as shown below:

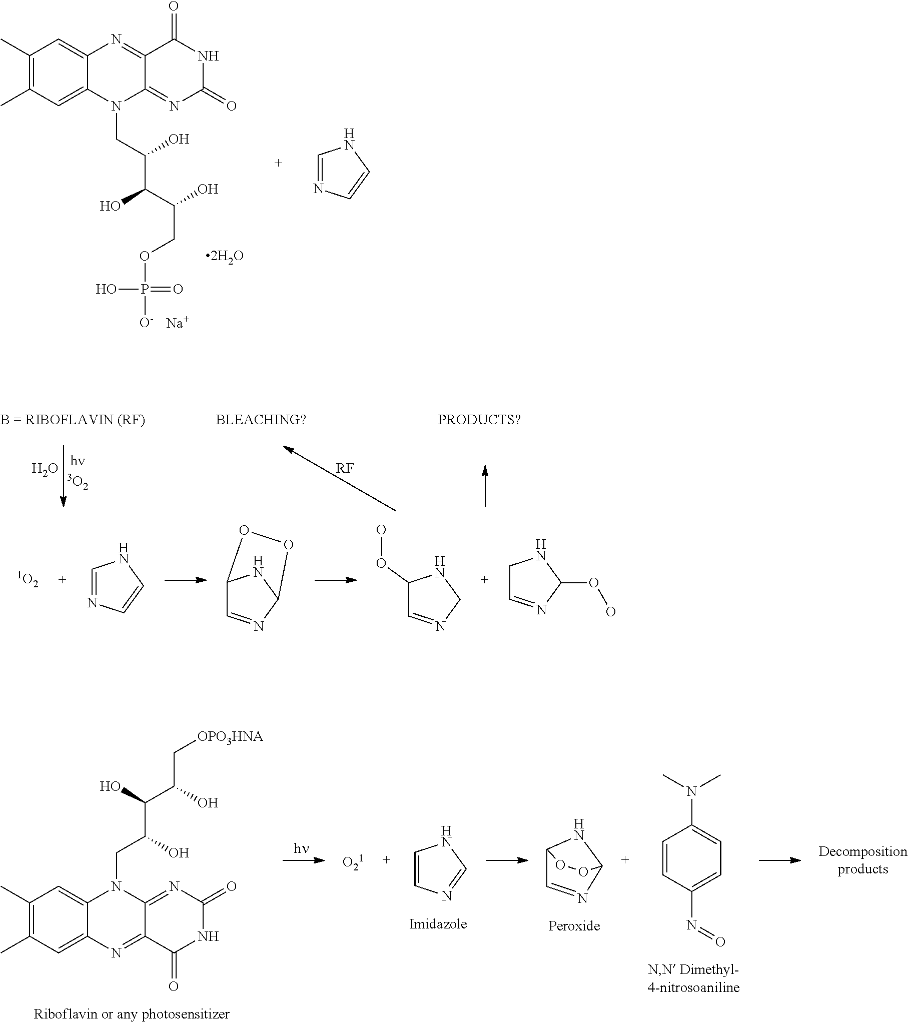

Reaction of a photosensitizer with imidazole in the presence of oxygen to form an endoperoxide and the subsequent bleaching of N,N-Dimethyl-4-nitrosoaniline by the various peroxides formed.

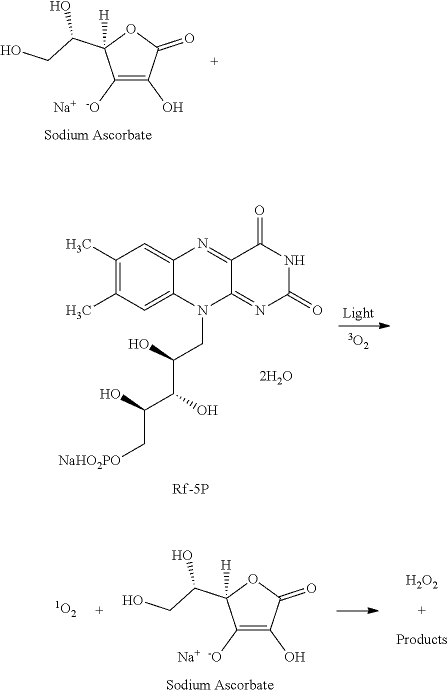

Reaction of sodium ascorbate with the photosensitizer, riboflavin5-phosphate in the presence of oxygen (Rb-5P) to form hydrogen peroxide.

The amount of such light energy that is required is typically greater than about 50 mW per square meter of solution surface exposed to said light, greater than about 1 milliwatt per square meter of solution surface exposed to said light, or even greater than 1×10-2 watts/square meter of solution surface exposed to said light. In some embodiments, the amount of light energy can be greater than about 100 mW/cm² and less than about 6 W/cm². In some embodiments, the amount of light energy can be about 200 mW/m². Said light energy may be provided by any suitable source, as described previously, including but not limited a light source located in a domestic appliance, said appliance being suitable for cleaning fabrics.

In addition to riboflavin, the LACC of the present invention can comprise imidazole, furans, and/or sodium ascorbate, and in some embodiments may also comprise one or more additional actives.

### Catalysts

In some embodiments, soluble active agents can include one or more metal catalysts. In some embodiments, the metal catalyst can include one or more of dichloro-1,4-diethyl-1,4,8,11-tetraazabicyclo[6.6.2]hexadecane manganese(II); and dichloro-1,4-dimethyl-1,4,8,11-tetraazabicyclo[6.6.2]hexadecane manganese(II). In some embodiments, the non-metal catalyst can include one or more of 2-[3-[(2-hexyldodecyl)oxy-2-(sulfooxy)propyl]-3,4-dihydroisoquinolinium, inner salt; 3,4-dihydro-2-[3-[(2-pentyldecyl)oxy]-2-(sulfooxy)propyl]isoquinolinium, inner salt: 2-[3-[(2-butyldecyl)oxy]-2-(sulfooxy)propyl]-3,4-dihydroisoquinolinium, inner salt; 3,4-dihydro-2-[3-(octadecyloxy)-2-(sulfooxy)propyl]isoquinolinium, inner salt; 2-[3-(hexadecyloxy)-2-(sulfooxy)propyl]-3,4-dihydroisoquinolinium, inner salt; 3,4-dihydro-2-[2-(sulfooxy)-3-(tetradecyloxy)propyl]isoquinolinium, inner salt: 2-[3-(dodecyloxy)-2-(sulfooxy)propyl]-3,4-dihydroisoquinolinium, inner salt; 2-[3-[(3-hexyldecyl)oxy]-2-(sulfooxy)propyl]-3,4-dihydroisoquinolinium, inner salt; 3,4-dihydro-2-[3-[(2-pentylnonyl)oxy]-2-(sulfooxy)propyl]isoquinolinium, inner salt; 3,4-dihydro-2-[3-[(2-propylheptyl)oxy]-2-(sulfooxy)propyl]isoquinolinium, inner salt; 2-[3-[(2-butyloctyl)oxy]-2-(sulfooxy)propyl]-3,4-dihydroisoquinolinium, inner salt; 2-[3-(decyloxy)-2-(sulfooxy)propyl]-3,4-dihydroisoquinolinium, inner salt; 3,4-dihydro-2-[3-(octyloxy)-2-(sulfooxy)propyl]isoquinolinium, inner salt; and 2-[3-[(2-ethylhexyl)oxy]-2-(sulfooxy)propyl]-3,4-dihydroisoquinolinium, inner salt.

### Activators

In some embodiments, LACC can include one or more activators. In some embodiments, the activator can include one or more of tetraacetyl ethylene diamine (TAED); benzoylcaprolactam (BzCL); 4-nitrobenzoylcaprolactam; 3-chlorobenzoylcaprolactam; benzoyloxybenzenesulphonate (BOBS); nonanoyloxybenzene-sulphonate (NOBS); phenyl benzoate (PhBz); decanoyloxybenzenesulphonate (C₁₀-OBS); benzoylvalerolactam (BZVL); octanoyloxybenzenesulphonate (C₈-OBS); perhydrolyzable esters; 4-[N4-(nonaoyl) amino hexanoyloxy]-benzene sulfonate sodium salt (NACA-OBS); dodecanoyloxybenzenesulphonate (LOBS or C₁₂-OBS); 10-undecenoyloxybenzenesulfonate (UDOBS or C₁₁-OBS with unsaturation in the 10 position); decanoyloxybenzoic acid (DOBA); (6-oclanamdocaproyl)oxybenzenesulfonate; (6-nonanamidocaproyl) oxybenzenesulfonate; and (6-decanamidocaproyl)oxybenzenesulfonate.

### Peroxy-Carboxylic Acids

In some embodiments, LACC can include one or more preformed peroxy carboxylic acids. In some embodiments, the peroxy carboxylic acids can include one or more of peroxymonosulfuric acids; perimidic acids; percabonic acids; percarboxilic acids and salts of said acids; phthalimidoperoxyhexanoic acid; amidoperoxyacids; 1,12-diperoxydodecanedioic acid; and monoperoxyphthalic acid (magnesium salt hexahydrate), wherein said amidoperoxyacids may include N,N′-terephthaloyl-di(6-aninocaproic acid), a ionononylamide of either peroxysuccinic acid (NAPSA) or of peroxyadipic acid (NAPAA), or N-nonanoylaminoperoxycaproic acid (NAPCA).

In some embodiments, water-based and/or water-soluble benefit agent can include one or more diacyl peroxide. In some embodiments, the diacyl peroxide can include one or more of dinonanoyl peroxide, didecanoyl peroxide, diundecanoyl peroxide, dilauroyl peroxide, and dibenzoyl peroxide, di-(3,5,5-trimethyl hexanoyl) peroxide, wherein said diacyl peroxide can be clatharated.

### Enzymes

In some embodiments, LACC can include one or more enzymes. In some embodiments, the enzyme can include one or more of peroxidases, proteases, lipases, phospholipases, cellulases, cellobiohydrolases, cellobiose dehydrogenases, esterases, cutinases, pectinases, mannanases, pectate lyases, keratinases, reductases, oxidases, phenoloxidases, lipoxygenases, ligninases, pullulanases, tannases, pentosanases, glucanases, arabinosidases, hyaluronidase, chondroitinase, laccases, amylases, dnases, and Photoactive enzymes such as monooxygenase.

### Sensate

In some embodiments, LACC can include one or more components that provide a sensory benefit, often called a sensate. Sensates can have sensory attributes such as a warming, tingling, or cooling sensation. Suitable sensates include, for example, menthol, menthyl lactate, leaf alcohol, camphor, clove bud oil, eucalyptus oil, anethole, methyl salicylate, eucalyptol, cassia, 1-8 menthyl acetate, eugenol, oxanone, alpha-irisone, propenyl guaethol, thymol, linalool, benzaldehyde, cinnamaldehyde glycerol acetal known as CGA, Winsense WS-5 supplied by Renessenz-Synmrise, Vanillyl butyl ether known as VBE, and mixtures thereof.

In some embodiments, the sensate comprises a coolant. The coolant can be any of a wide variety of materials. Included among such materials are carboxamides, menthol, ketals, diols, and mixtures thereof. Some examples of carboxamide coolants include, for example, paramenthan carboxyamide agents such as N-ethyl-p-menthan-3-carboxamide, known commercially as “WS-3”, N,2,3-trimethyl-2-isopropylbutanamide, known as “WS-23,” and N-(4-cyanonethylphenyl)-ρ-menthanecarboxamide, known as G-180 and supplied by Givaudan. G-180 generally comes as a 7.5% solution in a flavor oil, such as spearmint oil or peppermint oil. Examples of menthol coolants include, for example, menthol; 3-1-menthoxypropane-1,2-diol known as TK-10, manufactured by Takasago; menthone glycerol acetal known as MGA manufactured by Haarmann and Reimer; and menthyl lactate known as Frescolat® manufactured by Haarmann and Reimer. The terms menthol and menthyl as used herein include dextro- and levorotatory isomers of these compounds and racemic mixtures thereof.

In some embodiments, the sensate comprises a coolant selected from the group consisting of menthol; 3-1-menthoxypropane-1,2-diol, menthyl lactate; N,2,3-trimethyl-2-isopropylbutanamide; N-ethyl-p-menthan-3-carboxamide; N-(4-cyanomethylphenyl)-ρ-menthanecarboxamide, and combinations thereof. In further embodiments, the sensate comprises menthol; N,2,3-trimethyl-2-isopropylbutanamide.

### Surfactant

Detersive Surfactant: Suitable detersive surfactants include anionic detersive surfactants, non-ionic detersive surfactant, cationic detersive surfactants, zwitterionic detersive surfactants and amphoteric detersive surfactants and mixtures thereof. Suitable detersive surfactants may be linear or branched, substituted or un-substituted, and may be derived from petrochemical material or biomaterial. Preferred surfactant systems comprise both anionic and nonionic surfactant, preferably in weight ratios from 90:1 to 1:90. In some instances a weight ratio of anionic to nonionic surfactant of at least 1:1 is preferred. However, a ratio below 10:1 may be preferred. When present, the total surfactant level is preferably from 0.1% to 60%, from 1% to 50% or even from 5% to 40% by weight of the subject composition.

Anionic detersive surfactant: Anionic surfactants include, but are not limited to, those surface-active compounds that contain an organic hydrophobic group containing generally 8 to 22 carbon atoms or generally 8 to 18 carbon atoms in their molecular structure and at least one water-solubilizing group preferably selected from sulfonate, sulfate, and carboxylate so as to form a water-soluble compound. Usually, the hydrophobic group will comprise a C8-C22 alkyl, or acyl group. Such surfactants are employed in the form of water-soluble salts and the salt-forming cation usually is selected from sodium, potassium, ammonium, magnesium and mono-, with the sodium cation being the usual one chosen.

Anionic surfactants of the present invention and adjunct anionic cosurfactants, may exist in an acid form, and said acid form may be neutralized to form a surfactant salt which is desirable for use in the present compositions. Typical agents for neutralization include the metal counterion base such as hydroxides, e.g., NaOH or KOH. Further preferred agents for neutralizing anionic surfactants of the present invention and adjunct anionic surfactants or cosurfactants in their acid forms include ammonia, amines, oligoamines, or alkanolamines. Alkanolamines are preferred. Suitable non-limiting examples including monoethanolamine, diethanolamine, triethanolamine, and other linear or branched alkanolamines known in the art; for example, highly preferred alkanolamines include 2-amino-1-propanol, I-aminopropanol, monoisopropanolamine, or I-amino-3-propanol. Amine neutralization may be done to a full or partial extent, e.g. part of the anionic surfactant mix may be neutralized with sodium or potassium and part of the anionic surfactant mix may be neutralized with amines or alkanolamines.

Suitable sulphonate detersive surfactants include methyl ester sulphonates, alpha olefin sulphonates, alkyl benzene sulphonates, especially alkyl benzene sulphonates, preferably C10-1³ alkyl benzene sulphonate. Suitable alkyl benzene sulphonate (LAS) is obtainable, preferably obtained, by sulphonating commercially available linear alkyl benzene (LA3). Suitable LAB includes low 2-phenyl LAB, such as those supplied by Sasol under the tradename Isochem® or those supplied by Petresa under the tradenane Petrelab®, other suitable LAB include high 2-phenyl LAB, such as those supplied by Sasol under the tradename Hyblene®. A suitable anionic detersive surfactant is alkyl benzene sulphonate that is obtained by DETAL catalyzed process, although other synthesis routes, such as HF, may also be suitable. In one aspect a magnesium salt of LAS is used.

Suitable sulphate detersive surfactants include alkyl sulphate, preferably C₈₋₁₈ alkyl sulphate, or predominantly C₁₂ alkyl sulphate.

A preferred sulphate detersive surfactant is alkyl alkoxylated sulphate, preferably alkyl ethoxylated sulphate, preferably a C₈₋₁₈ alkyl alkoxylated sulphate, preferably a C₈₋₁₈ alkyl ethoxylated sulphate, preferably the alkyl alkoxylated sulphate has an average degree of alkoxylation of from 0.5 to 20, preferably from 0.5 to 10, preferably the alkyl alkoxylated sulphate is a C₈₋₁₈ alkyl ethoxylated sulphate having an average degree of ethoxylation of from 0.5 to 10, preferably from 0.5 to 5, more preferably from 0.5 to 3. The alkyl alkoxylated sulfate may have a broad alkoxy distribution or a peaked alkoxy distribution.

The alkyl sulphate, alkyl alkoxylated sulphate and alkyl benzene sulphonates may be linear or branched, including 2 alkyl substituted or mid chain branched type, substituted or un-substituted, and may be derived from petrochemical material or biomaterial. Preferably, the branching group is an alkyl. Typically, the alkyl is selected from methyl, ethyl, propyl, butyl, pentyl, cyclic alkyl groups and mixtures thereof. Single or multiple alkyl branches could be present on the main hydrocarbyl chain of the starting alcohol(s) used to produce the sulfated anionic surfactant used in the compositions of the invention. Most preferably the branched sulfated anionic surfactant is selected from alkyl sulfates, alkyl ethoxy sulfates, and mixtures thereof.

Alkyl sulfates and alkyl alkoxy sulfates are commercially available with a variety of chain lengths, ethoxylation and branching degrees. Commercially available sulfates include those based on Neodol alcohols ex the Shell company, Lial—Isalchem and Safol ex the Sasol company, natural alcohols ex The Procter & Gamble Chemicals company.

Other suitable anionic detersive surfactants include alkyl ether carboxylates.

Non-ionic detersive surfactant: Suitable non-ionic detersive surfactants are selected from the group consisting of: C₈-C₁₈ alkyl ethoxylates, such as, NEODOL® non-ionic surfactants from Shell; C₆-C₁₂ alkyl phenol alkoxylates wherein preferably the alkoxylate units are ethyleneoxy units, propyleneoxy units or a mixture thereof; C₁₂-C₁₈ alcohol and C6-C₁₂ alkyl phenol condensates with ethylene oxide/propylene oxide block polymers such as Pluronic® from BASF; alkylpolysaccharides, preferably alkylpolyglycosides; methyl ester ethoxylates; polyhydroxy fatty acid amides; ether capped poly(oxyalkylated) alcohol surfactants; and mixtures thereof.

Suitable non-ionic detersive surfactants are alkylpolyglucoside and/or an alkyl alkoxylated alcohol.

Suitable non-ionic detersive surfactants include alkyl alkoxylated alcohols, preferably C₈₋₁₈ alkyl alkoxylated alcohol, preferably a C₈₋₁₈ alkyl ethoxylated alcohol, preferably the alkyl alkoxylated alcohol has an average degree of alkoxylation of from 1 to 50, preferably from 1 to 30, or from 1 to 20, or from 1 to 10, preferably the alkyl alkoxylated alcohol is a C₈₋₁₈ alkyl ethoxylated alcohol having an average degree of ethoxylation of from 1 to 10, preferably from 1 to 7, more preferably from 1 to 5 and most preferably from 3 to 7. The alkyl alkoxylated alcohol can be linear or branched and substituted or un-substituted. Suitable nonionic surfactants include those with the trade name Lutensol® from BASF.

Cationic detersive surfactant: Suitable cationic detersive surfactants include alkyl pyridinium compounds, alkyl quaternary ammonium compounds, alkyl quaternary phosphonium compounds, alkyl ternary sulphonium compounds, and mixtures thereof.

Preferred cationic detersive surfactants are quaternary ammonium compounds having the general formula:

(R)(R₁)(R₂)(R₃)N⁺X⁻

wherein, R is a linear or branched, substituted or unsubstituted C₆₋₁₈ alkyl or alkenyl moiety, R₁ and R₂ are independently selected from methyl or ethyl moieties, R₃ is a hydroxyl, hydroxymethyl or a hydroxyethyl moiety, X is an anion which provides charge neutrality, preferred anions include: halides, preferably chloride; sulphate; and sulphonate.

Amphoteric and Zwitterionic detersive surfactant: Suitable amphoteric or zwitterionic detersive surfactants include amine oxides, and/or betaines. Preferred amine oxides are alkyl dimethyl amine oxide or alkyl amido propyl dimethyl amine oxide, more preferably alkyl dimethyl amine oxide and especially coco dimethyl amino oxide. Amine oxide may have a linear or mid-branched alkyl moiety. Typical linear amine oxides include water-soluble amine oxides containing one R1 C8-18 alkyl moiety and 2 R2 and R3 moieties selected from the group consisting of C1-3 alkyl groups and C1-3 hydroxyalkyl groups. Preferably amine oxide is characterized by the formula R1-N(R2)(R3) O wherein R1 is a C8-18 alkyl and R2 and R3 are selected from the group consisting of methyl, ethyl, propyl, isopropyl, 2-hydroxyethyl, 2-hydroxypropyl and 3-hydroxypropyl. The linear amine oxide surfactants in particular may include linear C10-C18 alkyl dimethyl amine oxides and linear C8-C12 alkoxy ethyl dihydroxy ethyl amine oxides.

Other suitable surfactants include betaines, such as alkyl betaines, alkylamidobetaine, amidazoliniumbetaine, sulfobetaine (INCI Sultaines) as well as Phosphobetaines.

Other suitable surfactants include Tween 20.

### Antimicrobial Compounds

In some embodiments, LACC can include an effective amount of a compound for reducing the number of viable microbes in the air or on inanimate surfaces. Antimicrobial compounds are effective on gram negative or gram positive bacteria or fungi typically found on indoor surfaces that have contacted human skin or pets such as couches, pillows, pet bedding, and carpets. Such microbial species include Klebsiella pneumoniae, Staphylococcus aureus, Aspergillus niger, Klebsiella pneumoniae, Steptococcus pyogenes, Salmonella choleraesuis, Escherichia coli, Trichophyton mentagrophytes, and Pseudomonoas aeruginosa. The antimicrobial compounds may also be effective at reducing the number of viable viruses such H1-N1, Rhinovirus, Respiratory Syncytial, Poliovirus Type 1, Rotavirus, Influenza A, Herpes simplex types 1 & 2, Hepatitis A, and Human Coronavirus.

Antimicrobial compounds suitable in the LACC can be any organic material which will not cause damage to fabric appearance (e.g., discoloration, coloration such as yellowing, bleaching). Water-soluble antimicrobial compounds include organic sulfur compounds, halogenated compounds, cyclic organic nitrogen compounds, low molecular weight aldehydes, quaternary compounds, dehydroacetic acid, phenyl and phenoxy compounds, or mixtures thereof.

A quaternary compound may be used. Examples of commercially available quaternary compounds suitable for use in the LACC are Barquat available from Lonza Corporation; and didecyl dimethyl ammonium chloride quat under the trade name Bardac® 2250 from Lonza Corporation.

The antimicrobial compound may be present in an amount from about 500 ppm to about 7000 ppm, alternatively about 1000 ppm to about 5000 ppm, alternatively about 1000 ppm to about 3000 ppm, alternatively about 1400 ppm to about 2500 ppm, by weight of the LACC.

### Preservatives

In some embodiments, LACC can include a preservative. The preservative may be present in an amount sufficient to prevent spoilage or prevent growth of inadvertently added microorganisms for a specific period of time, but not sufficient enough to contribute to the odor neutralizing performance of the LACC. In other words, the preservative is not being used as the antimicrobial compound to kill microorganisms on the surface onto which the LACC is deposited in order to eliminate odors produced by microorganisms. Instead, it is being used to prevent spoilage of the LACC in order to increase the shelf-life of the LACC.

The preservative can be any organic preservative material which will not cause damage to fabric appearance, e.g., discoloration, coloration, bleaching. Suitable water-soluble preservatives include organic sulfur compounds, halogenated compounds, cyclic organic nitrogen compounds, low molecular weight aldehydes, parabens, propane diol materials, isothiazolinones, quaternary compounds, benzoates, low molecular weight alcohols, dehydroacetic acid, phenyl and phenoxy compounds, or mixtures thereof.

Non-limiting examples of commercially available water-soluble preservatives include a mixture of about 77% 5-chloro-2-methyl-4-isothiazolin-3-one and about 23% 2-methyl-4-isothiazolin-3-one, a broad spectrum preservative available as a 1.5% aqueous solution under the trade name Kathon® CG by Rohm and Haas Co.; 5-bromo-5-nitro-1,3-dioxane, available under the tradename Bronidox L® from Henkel; 2-bromo-2-nitropropane-1,3-diol, available under the trade name Bronopol® from Inolex; 1,1′-hexamethylene bis(5-(p-chlorophenyl)biguanide), commonly known as chlorhexidine, and its salts, e.g., with acetic and digluconic acids; a 95:5 mixture of 1,3-bis(hydroxymethyl)-5,5-dimethyl-2,4-imidazolidinedione and 3-butyl-2-iodopropynyl carbamate, available under the trade name Glydant Plus® from Lonza; N-[1,3-bis(hydroxymethyl)2,5-dioxo-4-imidazolidinyl]-N,N′-bis(hydroxy-methyl) urea, commonly known as diazolidinyl urea, available under the trade name Germall® II from Sutton Laboratories, Inc.; N,N″-methylenebis{N′-[1-(hydroxymethyl)-2,5-dioxo-4-imidazolidinyl]urea}, commonly known as imidazolidinyl urea, available, e.g., under the trade name Abiol® from 3V-Sigma, Unicide U-13® from Induchem, Germall 115® from Sutton Laboratories, Inc.; polymethoxy bicyclic oxazolidine, available under the trade name Nuosept® C from Hills America; formal-dehyde; glutaraldehyde; polyaminopropyl biguanide, available under the trade name Cosmocil CQ® from ICI Americas, Inc., or under the trade name Mikrokill® from Brooks, Inc; dehydroacetic acid; and benzsiothiazolinone available under the trade name Koralone™ B-119 from Rohm and Hass Corporation; 1,2-Benzisothiazolin-3-one; Acticide MBS; Kathon CG/ICP.

Suitable levels of preservative are from about 0.0001 wt. % to about 0.5 wt. %, alternatively from about 0.0002 wt. % to about 0.2 wt. %, alternatively from about 0.0003 wt. % to about 0.1 wt. %, by weight of the LACC.

### Adjuvants

Adjuvants can be added to the LACC herein for their known purposes. Such adjuvants include, but are not limited to, water soluble metallic salts, including zinc salts, copper salts, and mixtures thereof; antistatic agents; insect and moth repelling agents; colorants; antioxidants; aromatherapy agents and mixtures thereof.

The compositions of the present invention can also comprise any additive usually used in the field under consideration. For example, non-encapsulated pigments, film forming agents, dispersants, antioxidants, essential oils, preserving agents, fragrances, liposoluble polymers that are dispersible in the medium, fillers, neutralizing agents, silicone elastomers, cosmetic and dermatological oil-soluble active agents such as, for example, emollients, moisturizers, vitamins, anti-wrinkle agents, essential fatty acids, sunscreens, and mixtures thereof can be added.

### Solvents

The composition can contain a solvent. Non-limiting examples of solvents can include ethanol, glycerol, propylene glycol, polyethylene glycol 400, polyethylene glycol 200, and mixtures thereof. In one example the composition comprises from about 0.5% to about 15% solvent, in another example from about 1.0% to about 10% solvent, and in another example from about 1.0% to about 8.0% solvent, and in another example from about 1% solvent to about 5% solvent. Suitable solvents also include Dowanol PNB-TR and DiPhB.

### Cleaning Emulsifiers

Compositions are often enhanced by the inclusion of emulsification aids that provide proper emulsification characteristics to remove soils without redeposition. Non limiting examples include Styleze-70, PEG8000 and Propyl Glycol Phenyl Ether. The preferred levels are between about 0.01 wt % and 1.0 wt %.

### Soil Capture Polymers

Compositions are often enhanced by the inclusion of soil capture polymers that aggregate to aid removal of soils from surfaces. Non limiting examples includes Mirapol HSC-300. The preferred levels are between about 0.01 wt % and 1.0 wt %.

### Anti-Foaming Agents

Compositions often require the inclusion of anti-foaming agents to prevent or minimize foaming during cleaning. None limiting agents include DC1410. The preferred levels are between about 0.01 wt % and 1.0 wt %.

### Methods of Application

The LACC can be applied to a fabric using conventional methods for cleaning formulations, such as spraying and exposing to light.

### Spray Dispenser:

A spray dispenser comprises a reservoir to accommodate the composition of the present disclosure and spraying means. Suitable spray dispensers include hand pump (sometimes referred to as “trigger”) devices, pressurized can devices, electrostatic spray devices, etc. Preferably the spray dispenser is non-pressurized and the spray means are of the trigger dispensing type. The reservoir is typically a container such as a bottle, more typically a plastic bottle.

The LACC is typically suitable for spraying from the spray dispenser onto the fabric surface to be treated (“direct application”). The composition preferably does not require any additional physical (e.g., manual rubbing) intervention.

The spray dispenser typically comprises a trigger lever which, once depressed, activates a small pump. The main moving element of the pump is typically a piston, housed inside a cylinder, with the piston pressing against a spring. By depressing the trigger, the piston is pushed into the cylinder and against the spring, compressing the spring, and forcing the composition contained within the pump out of a nozzle. Once the trigger lever is released, the spring pushes the piston back out, expanding the cylinder area, and sucking the composition from the reservoir, typically through a one-way valve, and refilling the pump. This pump is typically attached to a tube that draws the composition from the reservoir into the pump. The spray dispenser can comprise a further one-way valve, situated between the pump and the nozzle.

The nozzle comprises an orifice through which the composition is dispensed. The nozzle utilizes the kinetic energy of the composition to break it up into droplets as it passes through the orifice. Suitable nozzles can be plain, or shaped, or comprise a swirl chamber immediately before the orifice. Such swirl chambers induce a rotary fluid motion to the composition which causes swirling of the composition in the swirl chamber. A film is discharged from the perimeter of the orifice which typically results in dispensing the composition from the orifice as finer droplets.

Since such trigger-activated spray dispensers comprise a pump, the composition preferably is not pressurized within the reservoir and preferably does not comprise a propellant.

The spray dispenser can be a pre-compression sprayer which comprises a pressurized buffer for the composition, and a pressure-activated one-way valve between the buffer and the spray nozzle. Such precompression sprayers provide a more uniform spray distribution and more uniform spray droplet size since the composition is sprayed at a more uniform pressure. Such pre-compression sprayers include the Flairosol® spray dispenser, manufactured and sold by Afa Dispensing Group (The Netherlands) and the pre-compression trigger sprayers described in U.S. Patent Publication Nos. 2013/0112766 and 2012/0048959.

### Examples

### Example 1—Photobleach Test

### Photobleaching Experiments on Substrates

### Staining Solution Preparation:

Melanin (0.05-0.25 g) was dissolved in to 4 g of 1M NaOH and diluted with the 45 mL of 1% ethanol water solution. Melanin was purchased from SigmaAldrich, Saint Lois, MO.

The photobleaching experiments were carried out in a chamber with blue LED light source with a peak wavelength of 455 nm or 385 nm and a typical wattage at about 50 mW/m²-6 W/m². In each case a piece of Polyethylene coupon (12×12 cm roughly) is cut and a picture of the blank substrate is taken, subsequently the substrate is stained with 30 uL melanin concentration (0.1-0.5%) in quadruplicate. The stain was dried in air and a solution of Photobleach composition of the present invention either riboflavin 5-phosphate or thioxanthone (singlet oxygen generator); riboflavin 5-phosphate or thioxanthone with imidazole was dropwise added. The ratios of singlet oxygen generator to endo-/exo-peroxide generator was maintained at (9 uM of singlet oxygen generator: 9 mM of endo-exo-peroxide generator). The substrate is then left inside the chamber irradiated with blue or UV light of 50 mW/m²-6 W/m² for 30, 60 and 90 minutes. A final image is taken after the substrate has been exposed in the appropriate conditions and the chosen time period in the environmental chamber.

### Analysis

The photobleaching efficiency of various compositions of the present invention is quantified and is defined as

$$\% Efficiency = 100 \frac{Δ {E}_{0} - Δ {E}_{{t}_{n}}}{Δ {E}_{0}} \qquad ( 1 )$$

Where ΔE₀ and ΔEₙ are the color differences from the blank of the substrate at t=0 mins and t=time mins after being exposed to the light, as shown in FIG. 2. Color difference is defined as

$$Δ E = \sqrt{{( {R}_{2} - {R}_{1} )}^{2} + {( {G}_{2} - {G}_{1} )}^{2} + {( {B}_{2} - {B}_{1} )}^{2}} \qquad ( 2 )$$

Where ΔE is the color difference, R₁, R₂, G₁, G₂ and B₁, B₂ are the R G B values of the stain at times 1 and 2. An average of quadruplicate was used to calculate percent efficiency, as shown below in TABLE 1.

TABLE 1

| Ingredient | I | II | III | IV |
|---|---|---|---|---|
| Riboflavin 5-phospate | 0.005 | 0.005 | 0.005 | 0.005 |
| Imidazole | 0 | 3.35 | 16.75 | 33.5 |
| ethanol | 10 | 10 | 10 | 10 |
| water | 89.995 | 86.645 | 73.245 | 56.495 |
| % Efficiency | 20 | 37 | 33 | 55 |

Test conditions: 30 uL 1% Melanin solution stained spot, treated with 30 μL of bleaching solution (I-IV) treated with Blue light (455 nm), with 520 mW/m² for 75 min.

Photobleaching Experiment in Solution with Nitrosoaniline Marker

### General Solution Preparation

Solutions of photosensitizer (9-10 μM, 1 eq), nitrosoaniline visible marker (9-50 μM, 1-5 eq) and singlet oxygen quenching molecules (1-1000 equivalents) were prepared in a 10 mL volumetric flask either. Solvents for these studies were either 95:5 DI H₂O:Ethanol or pure ethanol. A starting UV-Vis spectrum was obtained as a baseline. Solutions were irradiated in a quartz cuvette using appropriate LED's at a measured distance to ensure reproducible results. Spectra were collected at intervals of 1 min, 5 min, 15 minutes, 30 minutes, and every 15 minutes until the nitrosoaniline visible marker was depleted. In cases where nitrosoaniline absorption signals were quenched faster, the spectra were collected as smaller intervals typically been 1-5 minutes.

### Stock Solution Preparation

### Riboflavin 5-Phosphate Sodium Salt Riboflavin 5-Phosphate Sodium Salt

Stock solutions of Riboflavin5-phosphate sodium salt were created by adding 2.2 mg of granulated Riboflavin 5-phosphate sodium salt Riboflavin 5-phosphate sodium salt into a 10 mL volumetric flask with a 95% H₂O: 5% ethanol to give a concentration of 0.46 mM. Adding 1 mL of the stock solution to another 10 mL was used to create a 46 μM solution. Adding 0.5 mL to a 25 mL volumetric flask gives a concentration of 9 μM.

### Thioxanthone

Stock solutions of thioxanthone were created by adding 3.5 mg of granulated thioxanthone into a 10 mL volumetric flask with pure ethanol to give a concentration of 2.3 mM. Adding 0.1 mL of the stock solution to another 10 mL was used to create a 23 μM solution. Adding 0.1 mL to a 25 mL volumetric flask gives a concentration of −9 μM.

### Sodium Ascorbate

Stock solutions of sodium ascorbate were created by adding 9.1 mg of granulated sodium ascorbate into a 10 mL volumetric flask with a 95% H₂O: 5% ethanol to give a concentration of 4.6 mM. Adding 0.1 mL of the stock solution to another 10 mL was used to create a 46 μM solution. Adding 0.05 mL to a 25 mL volumetric flask gives a concentration of −9 μM, which could be multiplied to give the number of equivalents needed for each study.

### Imidazole

Stock solutions of imidazole were created by adding 15.6 mg of granulated imidazole into a 10 mL volumetric flask with a 95% H₂O: 5% ethanol or pure ethanol to give a concentration of 2.3 mM. Adding 0.1 mL of the stock solution to another 10 mL was used to create a 23 μM solution. Adding 0.1 mL to a 25 mL volumetric flask gives a concentration of −9 μM, which could be multiplied to give the number of equivalents needed for each study.

N,N-Dimethyl-4-nitrosoaniline (Visible Marker)

Stock solutions of N,N-dimethyl-4-nitrosoaniline were created by adding 3.5 mg of granulated N,N-dimethyl-4-nitrosoaniline into a 10 mL volumetric flask with a 95% H₂O: 5% ethanol or pure ethanol to give a concentration of 2.3 mM. Adding 0.1 mL of the stock solution to another 10 mL was used to create a 23 μM solution. Adding 0.1 mL to a 25 mL volumetric flask gives a concentration of 9 μM, which could be multiplied to give the number of equivalents needed for each study.

### Light Sources

For some riboflavin decomposition experiments a home-built 450 nm LED was used to irradiate samples. The quartz cuvette was placed 11.5 cm away from the light source and the measured intensity was 1750 mW/m².

Some thioxanthone decomposition experiments were performed by using a home-built 365 nm LED. The quartz cuvette was located in the middle of 2 LED's approximately 2 cm from the sides of the cuvette. The light intensity was measured to be roughly 2000 mW/m² from each LED.

The last light source used in these experiments was a variable power LED array from LuzChem Research Inc. Samples loaded in quartz cuvettes were placed 4 cm away from the bulb and intensities were ranged from 2000-6000 mW/m2.

### UV-Visible Spectroscopy

A JASCO V-750 spectrophotometer (from JASCO Easton, Maryland, USA) was used for recording UV-Vis spectra. Quartz cuvettes were used for sample irradiations as they have an optical window that allows for spectra to be collected as low as 200 nm. IgorPro8 software was used for plotting and processing the data.

### Electrochemical Experiments to Determine Hydrogen Peroxide Formation

### Reagents

5-phosphate sodium salt, hydrogen peroxide and sodium ascorbate were made in 20 mM HEPES buffer, with an adjusted pH of 7.40±1.

### Carbon Fiber Microelectrodes

T-650 carbon fibers (Mitsubishi Chemical Carbon Fiber and Composites Inc., Sacramento, CA, USA), having a 7 mm diameter, were vacuum pooled into glass capillaries. These capillaries were then pulled vertically into two electrodes using a Narishige PE-22 electrode puller (Narishige Corp., Tokyo, Japan). These electrodes are cylindrical leaving fibers that are exposed to be cut under microscope to a length of 100-150 mm from the glass seal with a scalpel. Before use each electrode was soaked for 10 minutes in isopropyl alcohol to remove any debris that could impede detection. To create the electrical connection the electrodes were backfilled with 1M KCl.

### Fast Scan Cyclic Voltammetry

A potentiostat, WaveNeuro, with a 1-MΩ headstage (Pine Instruments, Durham, NC) was used to apply potentials to the electrode at fast-scan rates. For data analysis and acquisition a high-definition cyclic voltammetry (HDCV) software (University of North Carolina-Chapel Hill, courtesy of p. Mark Wightmnan) coupled with a PCIe-6363 multifunction I/O device, (National Instruments, Austin, TX) was used. Collected data was background subtracted to remove non-faradaic current, Calibration of electrodes was done using a Fusion 100 2-Channel Chemyx syringe pump (Chemyx Inc., Stafford, TX) with a flow rate of 1 mL/min.

The use of UV-Vis spectroscopy to measure the decay of the N, N′ Dimethyl-4-nitrosoaniline marker. This posed a significant issue for this study as the UV-Vis spectra for Riboflavin 5-phosphate sodium salt and N,N-dimethyl-4-nitrosoaniline were significantly overlapped.

Thioxanthone was chosen to be a surrogate Singlet Oxygen Generator (SOG) to replace Riboflavin 5-phosphate sodium salt for the imidazole and N,N-dimethyl-4-nitrosoaniline system. Thioxanthone is also a well-known singlet oxygen generator, with a triplet quantum yield of 0.56 in methanol solutions. This quantum yield places thioxanthone in the middle of the range of quantum yields reported for riboflavin and thus was used as a reasonable surrogate in replacement of Riboflavin 5-phosphate sodium salt for this experiment.

### Results

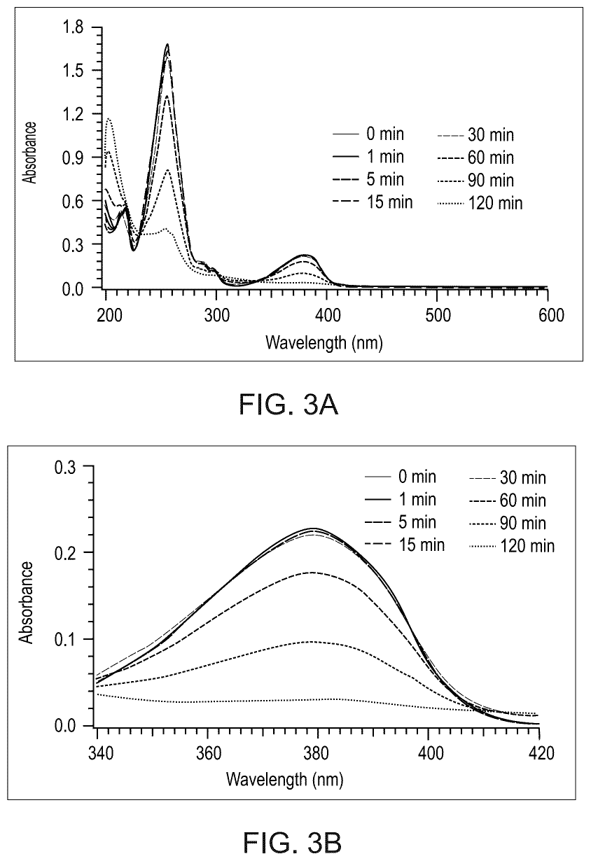

As can be seen in FIG. 3, depletion of thioxanthone 23 μM in ethanol with 365 nm irradiation was complete after two hours of irradiation.

Imidazole was added to the thioxanthone solution to test its bleaching effect. The thioxanthone concentration was held constant 23 μM and the imidazole concentration was varied between 23 μM and 230 μM to test the influence of additional imidazole on the system. The addition of imidazole increases the rate of thioxanthone degradation. In the 1:1 ratio of thioxanthone, the thioxanthone depletion experiences the same “ramping up” period as with thioxanthone alone, but the overall rate of decomposition is faster in comparison. In the case of the 1:10, ratio of thioxanthone to imidazole, the depletion of thioxanthone is uniform throughout, but still faster than with thioxanthone alone.

Next the bleaching of the N,N-dimethyl-4-nitrosoaniline marker by thioxanthone by itself tested. This time, the concentration of thioxanthone was varied between at 23 μM and 230 μM and the concentration of N,N-dimethyl-4-nitrosoaniline was held constant at 23 μM to test the effect of increased singlet oxygen on sensor depletion. When the concentration of thioxanthone to N,N-dimethyl-4-nitrosoaniline is the same, 23 μM, the results show a decrease in the N,N-dimethyl-4-nitrosoaniline and thioxanthone peaks. However, the decrease of thioxanthone is slowed in the presence of N,N-dimethyl-4-nitrosoaniline, suggesting the N,N-dimethyl-4-nitrosoaniline is acting to soak up the free singlet oxygen that is being generated thus preventing the loss of thioxanthone. After two hours of irradiation, neither the thioxanthone nor the N,N-dimethyl-4-nitrosoaniline is completely depleted. When the concentration of thioxanthone is increased to 230 μM in comparison to 23 μM of N,N-dimethyl-4-nitrosoaniline, the N,N-dimethyl-4-nitrosoaniline is rapidly depleted in 60 minutes. There is still some residual thioxanthone that takes another hour to fully deplete indicating continues bleaching or cleaning of soils. This suggests that increasing the thioxanthone concentration works effectively to produce faster depletion of the N,N-dimethyl-4-nitrosoaniline visible marker. It is also important to note that the depletion of N,N-dimethyl-4-nitrosoaniline alone by 365 nm irradiation yielded only negligible losses over 30 minutes.

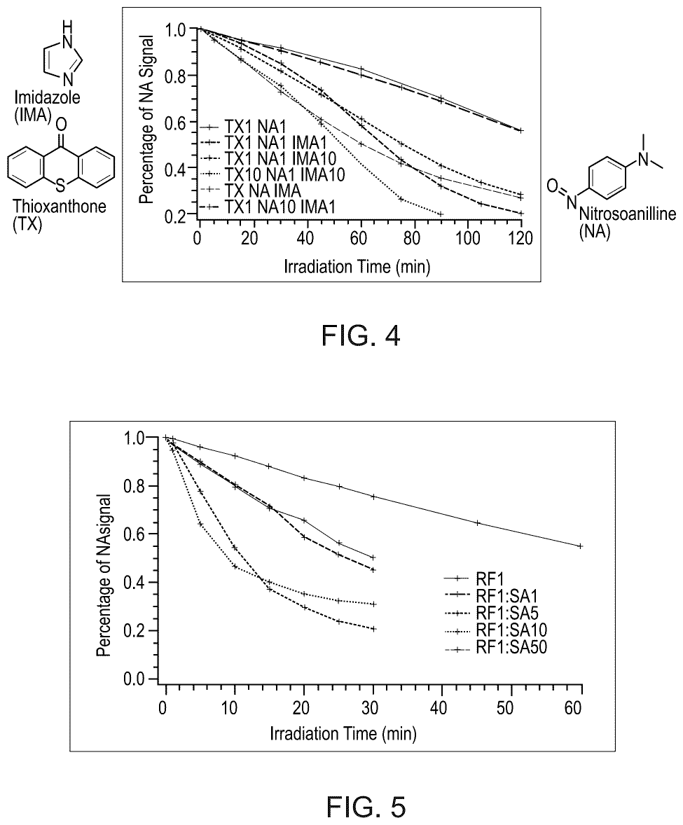

FIG. 4 shows a rate of decay of N,N-dimethyl-4-nitrosoaniline visible marker (NA) as detected by following it's absorbance at 424 nm. The concentrations of thioxanthone (TX), imidazole (IMA), and NA were varied to test the effect of concentration changes on the bleaching of NA. The standard concentration of the components, denoted by “1”, was 23 μM. “10” denotes 10 times this concentration or 230 μM.

FIG. 5 shows the impact of sodium ascorbate (SA) on Riboflavin 5-phosphate sodium salt (RF) degradation. Fractional RF Absorbance is the absorbance signal at 446 nm measured at an irradiation time divided by the absorbance signal at 446 nm at t=0. The concentration of RF was held constant at 46 μM, denoted by “1” and the concentration of sodium ascorbate (SA) was varied in multiples of 46 μM, denoted by “1”, “5”, “10”, “50” (i.e. 46 μM, 230 μM, 460 μM, and 2.3 mM).

To test the depletion of Riboflavin 5-phosphate sodium salt (RF) in presence of sodium ascorbate, experiments were run in triplicate using a 450 nm LED with a measured power of 1750 mW/m2. The concentrations of components were 46 μM r Riboflavin 5-phosphate sodium salt (RF) with no sodium ascorbate, a 1:1 ratio of Riboflavin 5-phosphate sodium salt (RF) and sodium ascorbate at 46 μM, and a 1:10 ratio of Riboflavin 5-phosphate sodium salt (RF) (46 μM) and sodium ascorbate (460 μM).

The results of these experiments showed the loss of Riboflavin 5-phosphate sodium salt (RF) signal intensity at 446 nm plotted as a fraction against irradiation time. Several time points were collected to ensure accurate measurements. The results of these experiments revealed that the loss of Riboflavin 5-phosphate sodium salt (RF) signal was very reproducible. Since these signals were reproducible, ratios of 1:5 Riboflavin 5-phosphate sodium salt (RF) (46 μM) to sodium ascorbate (230 μM) and 1:50 Riboflavin 5-phosphate sodium salt (RF) (46 μM) to sodium ascorbate (2.3 mM) were also studied.

The black trace in FIG. 5 is Riboflavin 5-phosphate sodium salt (RF) by itself, and shows roughly 50% loss of absorbance signal after 30 minutes of irradiation. The red line is a 1:1 ratio of Riboflavin 5-phosphate sodium salt (RF) and sodium ascorbate, and shows slightly more loss of Riboflavin 5-phosphate sodium salt (RF) absorbance signal after 30 minutes. However, during the first 15 minutes of irradiation, the loss of Riboflavin 5-phosphate sodium salt (RF) is roughly equal to the amount of Riboflavin 5-phosphate sodium salt (RF) loss without sodium ascorbate. This can likely be explained by the idea that H₂O₂ generated from sodium ascorbate needs to be accumulated before it reaches a concentration where it is able to adversely affect the Riboflavin 5-phosphate sodium salt (RF) photogenerator. In support of this idea, increasing the concentration of sodium ascorbate to 5× and 10× vs. the initial Riboflavin 5-phosphate sodium salt (RF) concentration, the deleterious effect on the absorbance signal of Riboflavin 5-phosphate sodium salt (RF) is demonstrated with about 50% signal loss in about 10 minutes. However, the interesting result is that at longer irradiation times the trial with 5× the concentration of sodium ascorbate outperforms the trial with 10× the concentration. At longer irradiation times for these concentrations, there becomes a point where the generated H₂O₂ begins to have a deleterious effect on the riboflavin and thus speeds up its decay as in the cases of 1× and 5× sodium ascorbate experiments. However, during the 10× sodium ascorbate experiment the rate of decay slows down in comparison to the 5× experiment. This could signify that the generated H₂O₂ may preferentially react with excess sodium ascorbate rather than Riboflavin 5-phosphate sodium salt (RF). This hypothesis of a preferential targeting of sodium ascorbate by H₂O₂ is supported by the linear and slow loss of Riboflavin 5-phosphate (RF) in the 50× sodium ascorbate experiment (maroon line in FIG. 5). The experiments at the lower concentrations of sodium ascorbate show that the amount of H₂O₂ generation is not adversely affected by increasing sodium ascorbate concentrations and grows with increasing sodium ascorbate concentration as expected.

### Odor Evaluation:

### General Method

Polycotton fabrics cut in pieces roughly (12×12 cm) were infused with 10 g of liquid smoke (Colgin Liquid Smoke) or Hot pot (brand) for 30 min in a sealed aluminum bag. The resulting odorous fabric was treated with formulations of the present invention having a singlet oxygen generator (SOG), or SOG plus Singlet Oxygen Quencher (SOQ) by spraying 1 gram of the formulations on each side the fabric as test substrate, for a total of 2 grams.

The treated fabrics were put in a chamber having a blue light LED or UV light source with a peak wavelength of 455 or 380 nm and were irradiated. A typical wattage at 50 mW/m² to about 6 W/m² was used for 30 min. Fabrics were taken out of the chamber, left to equilibrate in air for 30 min, and put in a sealed bag for panel evaluation. Six panelists were selected, and the panel was asked to rank order each one of the prototypes from 0 to 10, where in 0 indicates no smell and 10 indicates the level of smoke smell, the results of which are shown below in TABLE 2.

TABLE 2

| | Formulations | | | | |
|---|---|---|---|---|---|
| | I | II | III | IV | |
| | % | % | % | % | |
| Ingredients | Active | Active | Active | Active | V |
| Rb5P | 0.005 | 0.005 | 0.005 | 0.005 | |
| Imidazole | 0 | 3.35 | 16.75 | 33.5 | |
| Lupasol(Poly- | | | | | |
| ethyleneimine | | | | | |
| 50%) 60K Mw | | | | | |
| Ethanol | 10 | 10 | 10 | 10 | |
| Maleic Acid | | | | | |
| Bardac 2250J / | | | | | |
| Didecyl Dimethyl | | | | | |
| Ammonium Chloride / | | | | | |
| Lonza (50% DDAC) | | | | | |
| Water | 90 | 86.74 | 73.25 | 56.50 | |
| Smoked fabric | | | | | control |
| Odor | 3 | 2.4 | 0.2 | 0.2 | 10 |

As TABLE 2 depicts, there is significant reduction of odor as the amount of imidazole relative to riboflavin 5-phosphate sodium salt (RF) is increased. Odor detection of treated smoke odorous fabric become almost zero as the ratio of RF to imidazole reached 1:500, while untreated smoke odorous was rated 10.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A light activated cleaning composition comprising:
a photosensitizer; and
a singlet oxygen quenching molecule.

2. The light activated cleaning composition of claim 1, wherein the photosensitizer is at least one of substituted or unsubstituted Acetonaphthones, Acridines, Anthracenes, Anthraquinones, Anthrones, Azulenes, Benzophenones, Benzoquinones, Flavones, Camphoroquinone, Chrysenes, substituted 7-Dehydrocholesterols, Ergosterols, Fluorenes, s Fluorenones, Eosins, Fluoresceins, Phloxines, Rose Bengals, Erythrosins, Indoles, Naphthalenes, Phenanthrenes, Phenazines, Thionines, Azures, Toluidine Blue, Methylene Blues, Pyrenes, Quinoxalines, Retinols, Riboflavin, Rubrenes, Bacteriochlorophylls, Chlorophylls, Pheophytins, Pheophorbides, Protochlorophylls, proporphyrins, Fullerenes, Porphyrins, Metallo Porphyrins, Porphines, Rubrenes, and Phthalocyanines, phenosafranine, Thiamine hydrochloride, 2-((6,11-Dihydro-5H-dibenzo[b,e]azepin-6-yl)methyl)isoindoline-1,3-dione, Rivaroxaban Impurity 9, Warfarin sodium, Indomethacin, Dimethylaminoethyl acrylate benzyl chloride, Riboflavin 5-phosphate sodium salt, (3-Acrylamidopropyl)trimethylammonium Chloride, 2,2′-Bis(2-chlorophenyl)-4,4′,5,5′-tetraphenyl-1,2′-biimidazole, 7-(4-(Diethylamino)-2-ethoxyphenyl)-7-(1-ethyl-2-methyl-1H-indol-3-yl)furo[3,4-b]pyridin-5(7H)-one, Victoria Blue R, Ethalfluralin, N-(6-chloro-4-methylbenzo[d]thiazol-2-yl)-4-cyano-N-(2-morpholinoethyl)benzamide hydrochloride, Curcumin, Zincon, 2-Mercaptonicotinic acid, N-(cyclohexylcarbamothioyl)benzamide, Ninhydrin, Tolperisone, 3H-Indolium, 1,3,3-trimethyl-2-[2-(1-methyl-2-phenyl-1H-indol-3-yl)ethenyl]-, chloride (1:1), Chloropheniramine maleate, 3,3′-Carbonylbis(7-diethylaminocoumarin), Methyl (triphenylphosphoranylidene)acetate, 1 h-Pyrrole-2,5-dione, 1,1′-[1,2-ethanediylbis(thio-2,1-phenylene)]bis-1-Propanone, 1-(1,1′-biphenyl)-4-yl-2-methyl-2-(4-morpholinyl)-N-(2-chlorophenyl)-N′-{[2-(3-isopropoxyphenyl)-4-quinolinyl]carbonyl}thiourea, 2(1H)-Quinazolinethione, 4-(2-propen-1-ylamino)-, Benidipine Ethyl 2-amino-4-methyl-5-(4-nitrophenyl)thiophene-3-carboxylate, Irgacure 369 Monophosphothiamine, N-Benzyl Salbutamon Hydrochloride, Girard P reagent, Dimethyl(2-((2-methyl-1-oxoallyl)oxy)ethyl)(3-sulphopropyl)ammonium hydroxide, N-(2-Methyl-5-nitrophenyl)-4-(pyridin-3-YL)-1,3-thiazol-2-amine or their derivatives.

3. The light activated cleaning composition of claim 1, wherein the singlet oxygen quenching molecule comprises molecules having conjugated dienes.

4. The light activated cleaning composition of claim 3, wherein the molecules having conjugated dienes are at least one of substituted and unsubstituted imidazole, benzimidazole, indoles, pyrrole, furans, or guanine.

5. The light activated cleaning composition of claim 1, wherein the singlet oxygen quenching molecule is sodium ascorbate.

6. The light activated cleaning composition of claim 1, wherein the singlet oxygen quenching molecule comprises molecules having an allylic hydrogen.

7. The light activated cleaning composition of claim 2, comprising from about 10 ppm to about 100 ppm riboflavin-5-phosphate sodium salt, by weight of the light activated cleaning composition.

8. The light activated cleaning composition of claim 7, comprising from about 100 ppm to about 1000 ppm imidazole, by weight of the light activated cleaning composition.

9. The light activated cleaning composition of claim 8, wherein the ratio of riboflavin-5-phosphate to imidazole is from about 1:1 to about 1:1000.

10. The light activated cleaning composition of claim 1, comprising a polymer, wherein the polymer is at least one of polyamines or polyethyleneimine.

11. The light activated cleaning composition of claim 1, comprising a surfactant, wherein the surfactant is at least one of a nonionic surfactant, anionic surfactant, or cationic surfactant.

12. A method of cleaning a fabric, comprising:
applying a light activated cleaning composition comprising Riboflavin 5-phosphate sodium salt and imidazole to a fabric;
exposing the light activated cleaning composition to light; and
generating at least one of endoperoxide or exoperoxide or hydrogen peroxide.

13. The method of claim 12, wherein the light has a wavelength of from about 200 nm to about 600 nm.

14. The method of claim 12, where the intensity of light is from about 50 mW/m² to about 6 W/m².

15. The method of claim 12, wherein the light activated cleaning composition is exposed to light for at least 15 minutes.

16. The method of claim 12, wherein the method is done at a temperature of from about 15° C. to about 60° C.

17. The method of claim 12, wherein the ratio of Riboflavin 5-phosphate sodium salt to imidazole is from about 1:1 to about 1:1500.

18. A light activated cleaning composition comprising:
a photosensitizer; and
sodium ascorbate.

19. The light activated cleaning composition of claim 18, wherein the photosensitizer is riboflavin 5-phosphate sodium salt.

20. The light activated cleaning composition of claim 19, wherein the ratio of riboflavin 5-phosphate to sodium ascorbate is about 1 to 40.
